(19) United States
(12) Patent Application Publication
(10) Pub. No.: US 2026/0306089 A1
(43) Pub. Date: Oct. 8, 2026

(54) TISSUE TRACTION DEVICES

(71) Applicant: BOSTON SCIENTIFIC SCIMED, INC., Maple Grove, MN (US)

(72) Inventor: SCOTT EDWARD CORBEIL, Litchfield, NH (US)

(73) Assignee: BOSTON SCIENTIFIC SCIMED, INC., Maple Grove, MN (US)

(21) Appl. No.: 19/639,696

(22) Filed: Apr. 6, 2026

Related U.S. Application Data
(60) Provisional application No. 63/784,353, filed on Apr. 7, 2025.

(51) Int. Cl. A61B 17/02; A61B 17/00

(52) U.S. Cl. CPC A61B 17/0218; A61B 2017/00818; A61B 2017/00867; A61B 2017/00951

(57) ABSTRACT

The present disclosure relates to the field of tissue dissection. Specifically, the present disclosure relates to medical devices which lift target tissue during a dissection procedure to improve visualization of the target tissue and mitigate obstruction of dissection tools. In particular, the present disclosure relates to a tissue traction device which comprises a first tissue engagement portion configured to be coupled to a target tissue at an in-vivo target site, a second tissue engagement portion configured to be coupled to an anatomical wall opposing the target tissue, and a connecting structure located between and coupled to or integral with the first tissue engagement portion and the second tissue engagement portion.

### CROSS REFERENCE TO RELATED APPLICATIONS

The present application claims the benefit of US Provisional Patent Application Serial No. 63/784,353, filed Apr. 7, 2025, the disclosure of which is incorporated herein by reference.

### FIELD

The present disclosure relates to the field of medical devices. Specifically, the disclosure pertains to medical devices configured to provide tissue traction during endoscopic surgical procedures, such as endoscopic submucosal dissection (ESD) procedures.

### BACKGROUND

Surgical dissection of lesions from within narrow body passages, such as the digestive tract, may be inefficient and time-consuming due to poor target tissue visualization during the dissection procedure. This problem may be exacerbated during the procedure, as the partially dissected target tissue obstructs the working area to further decrease visibility and obstruct the dissection tools.

The present disclosure relates to the field of medical devices, specifically those configured to provide tissue traction during endoscopic surgical procedures. These devices enhance visualization and/or dissection of target tissues while mitigating obstruction of dissection tools.

Endoscopic surgical procedures enable minimally invasive treatment of various conditions within body passages, such as the gastrointestinal (GI) tract, by delivering specialized instruments through working channels of endoscopes to access and manipulate target tissues. Techniques like Endoscopic Mucosal Resection (EMR), Endoscopic Submucosal Dissection (ESD), and Pre-Oral Endoscopic Myotomy (POEM) allow for the en bloc resection of benign and early malignant lesions. Compared to open or laparoscopic surgery, these minimally invasive techniques typically offer faster recovery times but require precise tissue manipulation within confined anatomical spaces, necessitating a high degree of expertise.

ESD, in particular, is a minimally invasive procedure enabling en bloc resection of superficial GI tumors. It involves accessing and visualizing target tissue sites using an endoscope equipped with visualization capabilities, such as a light and/or camera. Through the endoscope's working channel, various medical instruments, including cutting devices, grasping tools, and tissue traction devices, are deployed to perform the dissection.

Tissue traction is critical during ESD procedures as it improves visualization of the submucosal layer, enabling accurate and efficient dissection. Maintaining proper tension on the target tissue during dissection is essential to enhance visibility and operational precision. However, surgical dissection within narrow body passages can be inefficient and time-consuming due to limited visibility. This challenge is often exacerbated by partially dissected tissue obstructing the working area and dissection tools.

Existing traction methods include tethered systems that use endoscopic clips attached to external filaments to retract or immobilize target tissue. Although effective in some cases, these systems can be limited in their ability to maintain or adjust tension on the target tissue. This limitation may obstruct visibility and interfere with accessory tools, potentially increasing procedure time, complexity, and the risk of complications, such as perforation or bleeding.

It is with these challenges in mind that the present disclosure introduces improved medical devices designed to optimize tissue traction during endoscopic procedures, thereby enhancing visualization, operational efficiency, and/or patient safety.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

The present disclosure provides tissue traction devices which are disposed in vivo and impart one or more forces on target tissue to maintain the target tissue under traction during a medical procedure such as a ESD procedure. As detailed herein, maintaining the target tissue such as a lesion under traction during a tissue dissection procedure (e.g., when dissecting tissue) can reduce complications, reduce an amount of time of the procedure, and/or improve visibility throughout the procedure. For instance, the tissue traction devices and methods herein can permit simultaneous cutting and precise tension control (e.g., at least radially within a body lumen) the tissue tractions devices. Moreover, unlike other approaches, the tissue traction devices and methods herein can be employed with an absence of clips and thus avoid issues (e.g., leaving foreign matter such as the clips behind in vivo) and/or complexities associated with employing multiple device exchanges. Thus, the devices and methods herein can yield faster and safer procedures (e.g., ESD procedures), while also addressing the limitations of current approaches.

In one aspect a tissue traction device is provided. The tissue traction device comprising: a first tissue engagement portion configured to be coupled to a target tissue at an in-vivo target site; a second tissue engagement portion configured to be coupled to an anatomical wall opposing the target tissue; and a connecting structure located between and coupled to or integral with the first tissue engagement portion and the second tissue engagement portion.

In some aspects, which may be used in conjunction with or as an alternative to other aspects herein, the tissue traction device further comprises a first coupling mechanism on the first tissue engagement portion, wherein the first coupling mechanism is configured to couple a surface of the first tissue engagement portion to the target tissue; and a second coupling mechanism on the second tissue engagement portion, wherein the second coupling mechanism is configured to couple a surface of the second tissue engagement portion to the anatomical wall opposing the target tissue.

In some aspects, which may be used in conjunction with or as an alternative to other aspects herein, wherein the first coupling mechanism, the second coupling mechanism, or both the first coupling mechanism and the second coupling mechanism are an adhesive.

In some aspects, which may be used in conjunction with or as an alternative to other aspects herein, wherein the first coupling mechanism, the second coupling mechanism, or both the first coupling mechanism and the second coupling mechanism are a mechanical anchor

In some aspects, which may be used in conjunction with or as an alternative to other aspects herein, wherein the mechanical anchor is a tine, fork, hook, finger, barb, or any combination thereof.

In some aspects, which may be used in conjunction with or as an alternative to other aspects herein, wherein the tissue traction device is configured to create tissue traction between the target tissue and the anatomical wall opposing the target tissue when the tissue traction device is coupled to the target tissue and the anatomical wall opposing the target tissue.

In some aspects, which may be used in conjunction with or as an alternative to other aspects herein, wherein: the first tissue engagement portion is a first planar patch; and the second tissue engagement portion is a second planar patch.

In some aspects, which may be used in conjunction with or as an alternative to other aspects herein, wherein the connecting structure is a substantially radially extending member that is coupled to opposing surfaces of the first planar patch and the second planar patch.

In some aspects, which may be used in conjunction with or as an alternative to other aspects herein, wherein the substantially radially extending member is formed of a shape memory material or an elastomeric material.

In some aspects, which may be used in conjunction with or as an alternative to other aspects herein, wherein the substantially radially extending member is included in a plurality of substantially radially extending members that are disposed at respective positions between the opposing surfaces of the first planar patch and the second planar patch.

In some aspects, which may be used in conjunction with or as an alternative to other aspects herein, wherein the substantially radially extending member comprises one or more substantially radially extending filaments or strips of material

In some aspects, which may be used in conjunction with or as an alternative to other aspects herein, wherein tissue traction device is a monolithic structure including the first tissue engagement portion, the second tissue engagement portion, and the connecting structure.

In some aspects, which may be used in conjunction with or as an alternative to other aspects herein, wherein the tissue traction device is formed of a shape memory material.

In some aspects, which may be used in conjunction with or as an alternative to other aspects herein, wherein: the first tissue engagement portion is located at a first end region of the monolithic structure; and the second tissue engagement portion is located at a second end region of the monolithic structure that opposes the first end region.

In some aspects, which may be used in conjunction with or as an alternative to other aspects herein, wherein the tissue traction device is a V-shaped, C-shaped, U-shaped, or Y-shaped monolithic structure.

In another aspect, a tissue traction device is provided. The tissue traction device comprising: a first tissue engagement portion configured to be coupled to a target tissue at an in-vivo target site; a first coupling mechanism coupled to or integral with the first tissue engagement portion; a second tissue engagement portion configured to be coupled to an anatomical wall opposing the target tissue; a second coupling mechanism coupled to or integral with the second tissue engagement portion; and an individual connecting structure that is integral with and located between the first and second tissue engagement portions.

In some aspects, which may be used in conjunction with or as an alternative to other aspects herein, wherein the tissue traction device is a monolithic structure including the first tissue engagement portion, the second tissue engagement portion, and the individual connecting structure.

In some aspects, which may be used in conjunction with or as an alternative to other aspects herein, wherein the monolithic structure is V-shaped.

In another aspect, a tissue traction device is provided. The tissue traction device comprising: a first tissue engagement portion configured to be coupled to a target tissue at an in-vivo target site; a first coupling mechanism coupled to or integral with the first tissue engagement portion; a second tissue engagement portion configured to be coupled to an anatomical wall opposing the target tissue; a second coupling mechanism coupled to or integral with the second tissue engagement portion; and a plurality of connecting structures coupled to and located between the first tissue engagement portion and the second tissue engagement portion, wherein each connecting structure of the plurality of connecting structures is a substantially radially extending filament or strip of material that is configured to impart a variable lifting force on the target tissue in response to insufflation of the target site.

In some aspects, which may be used in conjunction with or as an alternative to other aspects herein, wherein the substantially radially extending connecting structures are spaced apart along a longitudinal axis of the tissue traction device.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIGS. 1A-1B illustrate an example of a tissue traction device in a first configuration (FIG. 1A) and a second configuration (FIG. 1B), according to an embodiment of the present disclosure.

FIGS. 2A-2B illustrate another example of a tissue traction device in a first configuration (FIG. 2A) and a second configuration (FIG. 2B), according to an embodiment of the present disclosure.

FIG. 3 illustrates a method use of a tissue traction device for tissue dissection, according to an embodiment of the present disclosure.

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

Before the present disclosure is described in further detail, it is to be understood that the disclosure is not limited to the particular embodiments described, as such may vary. It is also to be understood that the terminology used herein is for the purpose of describing particular embodiments only, and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to medical devices and systems for dissecting tissues of the digestive system, it should be appreciated that such medical devices may be used to dissect tissues of the abdominal cavity, gastrointestinal system, thoracic cavity and the like. Moreover, a variety of medical procedures may benefit from presently disclosed medical devices, including, for example, Endoscopic Submucosal Dissection (ESD), Peroral Endoscopic Myotomy (POEM), cholecystectomy and Video-Assisted Thorascopic Surgery (VATS) procedures.

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to endoscopic systems and methods designed to provide traction within the gastrointestinal tract during a tissue dissection/resection procedure, it should be appreciated that such systems and methods may be used to manipulate a variety of tissues within a variety of different body lumens and/or body passages in conjunction with or independent of an endoscope.

As used herein, the singular forms “a,” “an,” and “the” are intended to include the plural forms as well, unless the context clearly indicates otherwise. It will be further understood that the terms “comprise”, “comprises”, and/or “comprising,” or “includes”, and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

As used herein, the terms “tissue traction”, “tissue tensioning”, “tissue lift”, “tissue retraction” or “retraction,” refers to the ability to control the position of a tissue during a dissection procedure. For example, tissue “traction” may allow a dissected portion of a target tissue to be immobilized and lifted away from the cutting plane to improve visualization of the remaining (i.e., non-dissected) target tissue, while also applying tension to the target tissue for more precise manipulation of the cutting element.

As used herein, the term “target tissue” refers to an unhealthy, diseased (i.e., cancerous, pre-cancerous etc.) or otherwise undesirable portion of tissue. A “target tissue” may also include tissues that are suspected of being unhealthy or diseased, but which require surgical removal for verification of their disease status by biopsy. It should be appreciated that surgical dissection of a “target tissue” typically includes removal of a portion of the surrounding healthy tissue along the “target tissue” margin to ensure complete removal and minimize the potential for metastasis of left behind or dislodged “target tissue” cells to other body locations.

In one embodiment, the present disclosure provides a tissue traction device which improves target tissue visibility and access during a dissection procedure by creating tissue traction between the engaged target lesion and opposing anatomical wall (e.g., on an opposing surface from a surface where the target lesion is located), including without occupying and/or obstructing a working channel of the endoscope.

The tissue traction devices herein include a first tissue engagement portion configured to be coupled to a target lesion at an in-vivo target site and a second tissue engagement portion configured to be coupled to an anatomical wall opposing the target lesion. The tissue traction devices further include a connecting structure located between and coupled to each of the first tissue engagement portion and the second tissue engagement portion. As mentioned, the tissue traction device is configured to create tissue traction between the engaged target lesion and opposing anatomical wall. The tissue traction device can be configured to move between a delivered configuration (e.g., where one or both of the first tissue engagement portion and the second tissue engagement portion are not coupled to tissue (e.g., a lesion at a target site in vivo) and a second (tissue tensioning) configuration where both the first tissue engagement portion and the second tissue engagement portion are coupled to tissue at the target site. For instance, the tissue traction device can be configured to impart opposing substantially radially extending forces (e.g., in a direction that is substantially normal to a central longitudinal axis extending along the vasculature in which tissue traction device is disposed. For instance, the tissue traction device can be configured to import forces that cause the first tissue engagement portion to contract radially toward the second tissue engagement portion when the tissue traction device is in the second (tissue tensioning) configuration. The radial contraction force can be a function of at least the type of material of the first tissue engagement portion, the second tissue engagement portion, and/or the connecting structure, as well as the relative orientation of the first tissue engagement portion, the second tissue engagement portion, and the connecting structure, as detailed herein.

In some embodiments, the connecting structure 116 can be an angled connecting structure. For instance, the tissue traction device can be manifested as a substantially V-shaped structure including an angled connecting structure that is configured to provide a contractive force between the first tissue engagement portion and the second tissue engagement portion which are located respectively on opposing ends of the V-shaped structure. In such embodiments, some or all of the V-shaped structure can be formed of a shape memory material which is configured (e.g., is heat-set) to predispose the V-shaped structure to a fixed or contracted configuration (e.g., once the first tissue engagement portion and the second tissue engagement are each coupled to tissue on opposing surfaces of an anatomical structure). The shape and size or the V-shaped structure and/or an angle of the angled connecting structure can be varied, for instance, depending on the anatomical structure diameter and geometry of the anatomical structure at a target site and/or depending on a desired amount of lifting force to be imparted on target tissue.

In some embodiments, the tissue traction devices herein such as the tissue traction device 111 include be manifested as two or more substantially planar patches or pads that are coupled together by the connecting structure 116 which is manifested as one or more substantially radially extending connecting structures such as filaments, strips of material, etc. The filaments or strips of material can be each be the same size and shape. Having each of the filaments or strips of material be the same size and shape (e.g., having a uniform width and length along each one of the filaments or strips of material can promote aspects herein such a promoting uniform application of force (e.g., a radial lifting force along a longitudinal length of a target lesion). However, in some embodiments, one or more of the filaments or strips of material can be a different size or shape.

In some embodiments, the tissue traction device 111 can consist of two or more substantially planar patches or pads that are connected by one or more substantially radially extending connecting structures, such as filaments or strips of material. These filaments or strips can be of uniform size and shape (e.g., having a consistent width and length), which can promote uniform application of force (e.g., a radial lifting force along the longitudinal length of a target lesion). However, in other embodiments, one or more of the filaments or strips of material may vary in size or shape.

The filaments or strips of material can be spaced apart along a longitudinal axis of the tissue traction device 111. For example, they can be substantially uniformly spaced along this axis to promote consistent force application. However, in some embodiments, one or more of the filaments or strips may be spaced at different intervals relative to adjacent filaments or strips, allowing for variable force distribution depending on the needs of the procedure.

In such embodiments, the connecting structure 116 can be a separate component coupled to each of the patches or an element that extends from and is integral with one or more (e.g., each of) the patches. In such cases, the connecting structure can be configured to contract or tighten relative to each of the patches, thereby imparting tissue tensioning on the target tissue, as detailed herein. For instance, the connecting structure can be formed of a shape memory material (e.g., nitinol) or an elastomer configured to contract. Additionally, the connecting structure can be configured to contract in response to the target site being insufflated, thereby permitting control over the degree of contraction and tissue tensioning, e.g., based on the degree of insufflation of the target site.

In some embodiments, the tissue traction devices herein can be coupled to a target site with a coupling mechanism such as an adhesive, a mechanical anchor, or a combination of an adhesive and a mechanical anchor. For instance, the first tissue engagement portion can be coupled to the target lesion with a first coupling mechanism in the form of an adhesive, a mechanical anchor, or a combination of an adhesive and a mechanical anchor. Similarly, the second tissue engagement portion can be coupled with a second coupling mechanism to the tissue on an opposing wall from the target tissue with an adhesive, a mechanical anchor, or a combination of an adhesive and a mechanical anchor. In some embodiments, the first tissue engagement portion and the second tissue engagement portion can be coupled to respective tissue with the same type of coupling mechanism (e.g., each with an adhesive or a mechanical anchor). However, in some embodiments, the first tissue engagement portion and the second tissue engagement portion can be coupled to respective tissue with a different type of coupling mechanism. For instance, in some embodiments the first tissue engagement portion can be coupled to the target tissue with a non-removably manner (e.g., with a given type of adhesive, a given type of mechanical anchor, and/or a given quantity of mechanical anchors), while the second tissue engagement portion can be coupled to an anatomical wall opposing the target lesion in a removable manner (e.g., with a different type of adhesive, a different type of mechanical anchor, and/or a different quantity of mechanical anchors e.g., that is less than a quantity of mechanical anchors employed in conjunction with the first tissue engagement portion).

In some embodiments, the adhesive and/or the mechanical anchors can be a biodegradable and/or biocompatible. For instance, the adhesive and/or the mechanical anchors can be biodegradable and biocompatible.

Examples of suitable adhesives include poly(lactic-co-glycolic acid) (PLGA) adhesives, chitosan adhesives, gelatin-based adhesives, and fibrin glue. In some examples, the amount of adhesive on the first tissue engagement portion 112 can be the same as that on the second tissue engagement portion 114. For instance, the adhesive on the first tissue engagement portion 112 can have the same dimensions (e.g., radial thickness and/or longitudinal length) as the adhesive on the second tissue engagement portion.

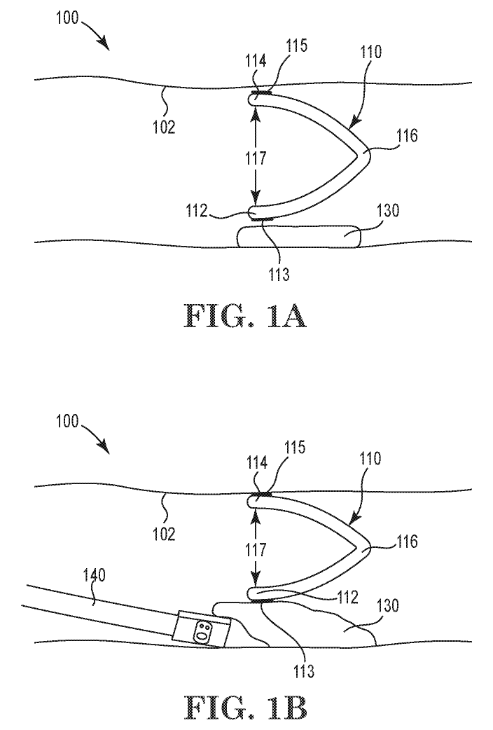

In some embodiments, the total surface area of the adhesive (i.e., a first coupling mechanism 113, as illustrated in FIGS. 1A-1B) on the first tissue engagement portion 112 can be substantially equal to the total surface area of the adhesive (i.e., a second coupling mechanism 115, as illustrated in FIGS. 1A-1B) on the second tissue engagement portion 114. Having equal adhesive surface areas on both engagement portions can help provide a substantially equal retention force between the tissue at the target site and each of the engagement portions, thereby promoting reliable and predictable coupling and/or decoupling of the first and second tissue engagement portions to the tissue.

However, in some embodiments, the adhesive surface area on the first tissue engagement portion 112 can be greater than that on the second tissue engagement portion. A larger adhesive surface area on the first tissue engagement portion can help ensure that the adhesive remains coupled to all or a majority of the target tissue (e.g., even after dissection) and can also facilitate the decoupling of the adhesive on the second tissue engagement portion 114 from the target site after dissection.

In some embodiments, the adhesive on both engagement portions may be of the same type. However, in other embodiments, different types of adhesives may be used. For instance, the adhesive on the first tissue engagement portion 112 could have a greater holding force or adhesion than that on the second tissue engagement portion 114. A stronger adhesive on the first tissue engagement portion can help ensure that the adhesive stays attached to the majority of the target tissue even after dissection, while also promoting decoupling of the adhesive on the second tissue engagement portion 114 from the target site.

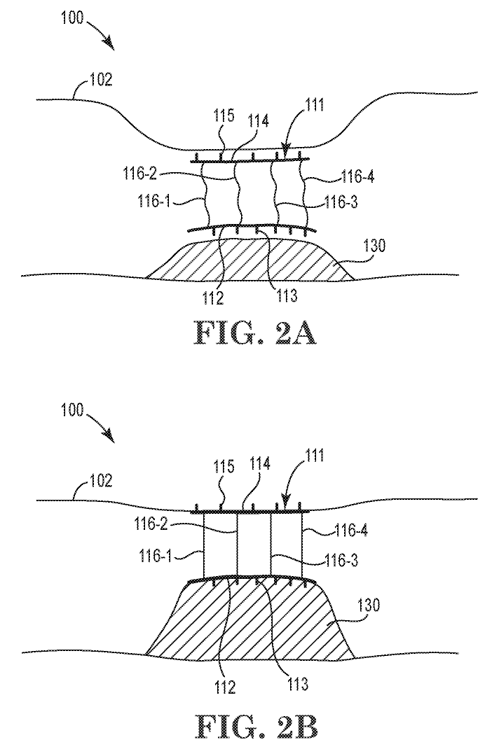

Examples of suitable mechanical anchors include wire prongs or tines, wire forks, fingers or hooks, wire ends and/or loops, or barbs. In addition, or alternatively to the adhesive and/or mechanical anchors, the first tissue engagement portion 112 and the second tissue engagement portion 114 may beneficially include sharpened or pointed tips to facilitate tissue penetration. In some embodiments, the coupling mechanisms 113, 115 (e.g., an adhesive or mechanical anchor) can be located on the outer (radial most) surfaces of the first tissue engagement portion 112 and the second tissue engagement portion 114. For example, the coupling mechanisms 113, 115 can be manifested as mechanical anchors located on the outer surfaces of the first tissue engagement portion 112 and the second tissue engagement portion, respectively (e.g., as illustrated in FIGS. 2A-2B). In such embodiments, the mechanical anchors can extend a distance radially outward relative to the longitudinal axis of the tissue traction device and/or a longitudinal axis of an anatomical structure in which the tissue traction device is disposed to permit the mechanical anchors to contact and embed a distance within tissue (e.g., a target lesion) at the target site. Any quantity of mechanical anchors and/or combinations of different types of mechanical anchors can be employed.

In one embodiment, these (and other) tissue engagement anchor configurations may comprise a shape memory material, e.g., nitinol, which is heat-set to point downwards when deployed but may be constrained in a straight position within a delivery catheter prior to deployment. When embedded within the target tissue, the first tissue engagement portion 112 exerts at least upward (e.g., lifting) and, optionally, outward (e.g., longitudinal) retraction forces into the target tissue, while simultaneously driving or pressing the second tissue engagement portion 114 of the tissue traction device 110 downward against the target tissue surface, e.g., when the tissue traction device 110 is oriented in the configuration illustrated in FIG. 1B. That is, the first tissue engagement portion 112 is configured to impart at least a tissue lifting force on the target tissue 130 at the same time that the second tissue engagement portion 114 provides an opposing force on the opposing anatomical wall 102. While various references are made herein to a first and/or second tissue engagement portions, upward, downward, it is understood that the first and second tissue engagement portions are relative terms and/or that the first and second tissue engagement portions are interchangeable elements that do not convey a particular orientation or geometry of the structures herein.

FIGS. 1A-1B illustrate a tissue traction device in a first configuration (FIG. 1A) and a second configuration (FIG. 1B), according to an embodiment of the present disclosure. As illustrated in FIGS. 1A-1B, in one embodiment, a tissue traction device 110 is disposed at a target site in an anatomical structure 100 in vivo. As illustrated in FIG. 1A, the tissue traction device 110 is configured in a first configuration. In the first configuration the first and second tissue engagement portions are not coupled to tissue adjacent thereto. While not illustrated, the tissue traction device 110 can be delivered in a delivery configuration where the tissue traction device has a smaller radial profile than the radial profile of the tissue traction device 110 when configured in the first configuration as illustrated in FIG. 1A.

As illustrated in FIG. 1A, in some embodiments the first tissue engagement portion 112 and the second tissue engagement portion 114 can be spaced a distance 117 (e.g., radially) apart. However, while FIG. 1A illustrates the presence of the distance 117 between opposing inner surface of the first tissue engagement portion 112 and the second tissue engagement portion 114, in some embodiments the first tissue engagement portion 112 and the second tissue engagement portion 114 can be in direct contact (e.g., the distance 117 is zero) when the tissue traction device is in a delivery configuration and when the tissue traction device is in a first configuration.

As mentioned, the tissue traction device can include a first tissue engagement portion 112, a second tissue engagement portion 114, and a connecting structure located between and coupled to, or integral with, the first tissue engagement portion 112 and the second tissue engagement portion 114. For example, the first tissue engagement portion 112 can be configured to be coupled to the target lesion 130 at an in-vivo target site, the second tissue engagement portion 114 can be configured to be coupled to an anatomical wall 102 opposing the target lesion, and a shape memory connecting structure 116 is between and integral with the first tissue engagement portion 112 and the second tissue portion 114. In such embodiments, the shape memory connecting structure 116 can be configured to contract inwardly relative to both the first tissue engagement portion 112 and the second tissue engagement member 114.

In some embodiments, the tissue traction devices herein are a monolithic structure including the first tissue engagement portion, the second tissue engagement portion, and the connecting structure. For instance, the first tissue engagement portion 112, the second tissue engagement portion 114, and the connecting structure 116 can form a monolithic structure, as illustrated in FIGS. 1A-1B. The first tissue engagement portion 112 and the second tissue engagement portion 114 can be located on opposing ends of the monolithic structure. For instance, the first tissue engagement portion 112 can be a first end region of the monolithic structure and the second tissue engagement portion 114 can be a second end region of the monolithic structure that opposes the first end region of the monolithic structure. The monolithic structure can be made of the same type of material and can have a uniform or varied diameter along its longitudinal length. For example, the monolithic structure can be formed entirely or at least partially of a shape memory material (e.g., nitinol) that is configured (e.g., heat set) to form a bilateral strip of material having bilateral symmetry such as a substantially V-shaped structure, as illustrated in FIGS. 1A-1B. For instance, the arms or branches of the V-shaped structure of the tissue traction device 110 on which the first tissue engagement portion 112 and the second tissue engagement portion 114 are disposed can be the same shape and size. The bilateral symmetry (e.g., the V-shaped structure) of the tissue traction device 110 can promote aspects described herein, such as readily permitting contact with and coupling to tissue adjacent to the first tissue engagement portion 112 and the second tissue engagement portion and/or providing a substantially radial tissue tensioning force on the target tissue 130. Imparting this substantially radially extending tissue tensioning force on the target tissue can yield an enhanced degree of tissue lift of the target lesion relative to adjacent tissue, compared to other approaches that may couple a tissue tensioning member to the adjacent tissue rather than to tissue on the opposing surface of the lumen. However, in some embodiments the arms or branches of the V-shaped structure on which the first tissue engagement portion 112 and the second tissue engagement portion 114 are disposed can be a different shape and/or a different size.

While FIGS. 1A-1B illustrate the tissue traction device 110 is a substantially V-shaped monolithic member (i.e., has a V-shaped form factor), the present disclosure is not so limited. For instance, other shapes of the monolithic tissue traction devices herein such as a C-shape, U-shape, Y-shape, etc. are possible and/or the tissue traction devices herein can be formed of two or more elements that are coupled together, as described herein, rather than being a monolithic tissue traction device (e.g., the monolithic tissue traction device 110).

As illustrated in FIG. 1A, the tissue traction device 110 is not in contact with the target tissue 130 nor an anatomical wall 100. However, the tissue traction device 110 can be placed in contact with tissue by application of force (e.g., with an endoscope or a tool inserted via a lumen of the endoscope) to the proximal end or other portion of the tissue traction device 110. For instance, a tool can be inserted through a lumen of the endoscope and can contact the first tissue engagement portion 112, the second tissue engagement portion 114, and/or the connecting structure 116 to cause the first tissue engagement portion 112 and the second tissue engagement portion 114 to move apart radially. This is merely an example and other methods of contacting the first tissue engagement portion 112 and the second tissue engagement portion 114 with tissue and/or anatomical wall are possible. The amount of force and/or a duration of the application of force to contact the first tissue engagement portion 112 and the second tissue engagement portion 114 with the tissue and/or anatomical wall can vary. For instance, the amount of force and/or the duration of the application of force can vary depending geometry of the target lesion, geometry of the opposing anatomical surface, a type and/or quantity of a coupling mechanism(s) on the first tissue engagement portion 112 and the second tissue engagement portion 114, etc. In any case, the application of force can result in the first tissue engagement portion 112 and the second tissue engagement portion 114 being coupled to respective opposing surfaces of the anatomic wall 100. For instance, the first tissue engagement portion 112 can be coupled to the target tissue 130 and the second tissue engagement portion 114 can be coupled to an opposing anatomical wall (a radially opposed wall relative to the target tissue 130.

It should be appreciated that the “force” stored within such materials when in the constrained configuration (e.g., when the first tissue engagement portion 112 and the second tissue engagement portion 114 are coupled to target tissue/anatomical walls opposing the target tissue) allows the tissue traction device 110 to apply and maintain constant upward (e.g., radial) lifting force against the target tissue 130 in which the first tissue engagement portion is coupled to. The natural tendency of the tissue traction device to move (i.e., contract the first tissue engagement portion 112 radially toward the second tissue engagement portion 114) allows the target tissue to be lifted or elevated above the cutting plane such that the non-dissected target tissue portion may be more easily visualized and more efficiently excised.

For instance, as illustrated in FIG. 1B, a distance 117 between the first tissue engagement portion 112 and the second tissue engagement portion 114 can remain substantially the same as the distance between the first tissue engagement portion 112 and the second tissue engagement portion 114 illustrated in FIG. 1A. However, given that first tissue engagement portion 112 is coupled to the target tissue 130 and the second tissue engagement portion 114 is coupled to an opposing anatomical wall 102 of the anatomic wall 100, the target tissue 130 can have a larger radial profile than a radial profile of the target tissue in FIG. 1A. As mentioned, tensioning the target tissue 130 e.g., as illustrated in FIG. 1B can desirably ease visibility and/or dissection of the target tissue 130.

For instance, with continued reference to FIG. 1B, a cutting element 140 may then be advanced through a working channel of an endoscope (not illustrated) to dissect the target tissue 130 (e.g., while lifted or under substantially radial tension imparted by the tissue traction device 110) from the surrounding healthy tissue. Due to the configuration of the tissue traction device 110 as detailed herein, the target tissue 130 can desirably remain under tension or lifted until a majority or all the target tissue 130 is dissected. Once the target tissue 130 is fully dissected, the target tissue may be removed for biopsy through a working channel of the endoscope using a suitable grasping element (not depicted). Alternatively, the target tissue 130, once dissected, may be allowed to remain within the body passage for removal by the body's natural course.

For instance, FIGS. 2A-2B illustrate another example of a tissue traction device 111 that is formed at least in part of a plurality of planar patches which are coupled together via one or more connecting structures. That is, FIGS. 2A-2B illustrate the tissue traction device 111 in a first configuration (FIG. 2A) and a second configuration (FIG. 2B), according to an embodiment of the present disclosure. As illustrated in FIGS. 2A-2B, in one embodiment, the tissue traction device 111 is disposed at a target site in an anatomical wall 100 in vivo. As illustrated in FIG. 2A, the tissue traction device 111 is configured in a first configuration. In the first configuration the first and second tissue engagement portions 112, 114 are not coupled to tissue adjacent thereto. While not illustrated, the tissue traction device 111 can be delivered in a delivery configuration where the tissue traction device 111 has a smaller radial profile than the radial profile of the tissue traction device 111 when configured in the first configuration as illustrated in FIG. 2A.

As mentioned, the tissue traction device 111 can include a first tissue engagement portion 112, a second tissue engagement portion 114, and a connecting structure 116 located between and coupled to, or integral with, the first tissue engagement portion 112 and the second tissue engagement portion 114. For example, the first tissue engagement portion 112 can be manifested as a first planar patch that is configured to be coupled to the 130 target lesion at an in-vivo target site and the second tissue engagement portion 114 can be manifested as a second planar patch that is be configured to be coupled to an anatomical wall 102 opposing the target lesion, as illustrated in FIGS. 2A-2B. While the first tissue engagement portion 112 and the second tissue engagement portion are described in some embodiments as being planar patches, the planar patches herein can exhibit a degree of curvature or twisting when coupled to tissue as a target site. For example, the planar patches herein can be formed of a shape-memory material or an elastomeric material can deform (e.g., curve or twist) to permit the planar patches herein to conform to the geometry of tissue at a target site.

The connecting structure 116 can be manifested as one or more substantially radially extending member that is coupled to opposing surfaces (e.g., inner surfaces that are most proximate to a central longitudinal axis of the body lumen in which the tissue traction device 111 is disposed) of the first planar patch and the second planar patch. For instance, the connecting structure 116 can be manifested as a plurality of radially extending members e.g., filaments or strips of material, etc., that are each coupled at respective locations to opposing surfaces of the first planar patch and the second planar patch, as illustrated in FIGS. 2A-2B. The opposing surfaces of the first planar patch and the second planar patch can be opposing inner surfaces (i.e., that are most proximate to a central longitudinal axis of an anatomical wall in which the tissue traction device 111 is disposed)) of the first planar patch and the second planar patch, as illustrated in FIGS. 2A-2B.

In some embodiments, the connecting structure 116 is an individual member. For instance, the connecting structure 116 can be manifested as the individual connecting structure illustrated in FIGS. 1A-1B. Stated differently, in some embodiments the tissue traction devices herein can include only one connecting structure 116. However, in some embodiments the connecting structure can be manifested as a plurality of connecting structures. For instance, the connecting structures herein can be manifested as a plurality of connecting structures 116-1, 116-2, 116-3, 116-4 (collectively referred to herein as connecting structures 116) as illustrated in FIGS. 2A-2B. As illustrated in FIGS. 2A-2B, each of the connecting structures 116 can be manifested as a respective filament that is substantially the same size and length as the other filaments forming the connecting structures. While FIGS. 2A-2B illustrate a given quantity (e.g., four) connecting structures 116, a type (e.g., a filament, a strip of material, etc.), and/or the quantity of connecting structures can be varied (e.g., increased or decreased).

In some embodiments, the connecting structure 116 (e.g., the filaments) can extend radially between the first planar patch and the second planar patch at about a 90 degree angle relative to each of the first planar patch and the second planar patch. For example, the first planar patch (i.e., the first tissue engagement portion 112) and the second planar patch (i.e., the second tissue engagement portion 114) can be located at substantially the same longitudinal position (albeit at different radial or circumferential positions) within the anatomical wall 100 and the connecting structure 116 can extend in a substantially linear manner between the first planar patch and the second planar patch (e.g., at least when the tissue traction device 111 is coupled to the target lesion and an anatomical wall opposing the target lesion). Having the connecting structure 116 (e.g., one or more filaments) extend between the first planar patch and the second planar patch at about a 90 degree angle can promote aspects herein such a promoting the lifting of the target tissue 130 in a substantially radial direction relative to a wall of the anatomical wall 100 in which the tissue traction device 111 is disposed. However, other angles are possible. For instance in some embodiments, the first planar patch and the second planar patch can be offset by a distance.

With continued reference to FIGS. 2A-2B, the connecting structure 116 can comprise one or more flexible elements formed of a shape memory material and/or an elastomeric material. Stated differently, the connecting structure 116 can be formed entirely or at least partially of a shape memory material (e.g., nitinol) and/or an elastic material. For instance, in some embodiments the connecting structure 116 can be formed be formed entirely of a shape memory material, an elastic material, or a combination of a shape memory material and an elastic material. As mentioned, the connecting structure 116 can be between and coupled to or integral with the first tissue engagement portion 112 and the second tissue portion 114. The connecting structure 116 can be configured to contract inwardly relative to both the first tissue engagement portion 112 and the second tissue engagement member 114. For instance, in some embodiments, the connecting structure 116 can be formed of an elastomeric material that is configured to contract responsive to insufflation of a target site in vivo where the target tissue 130 is located. As mentioned, the connecting structure 116 can be manifested as one or more filaments formed of an elastic material that are coupled to or integral with the first planar patch and the second planar patch can are configured to contract based on a degree of insufflation of the target site. Hence, an amount of force or lift imparted on the target tissue 130 can desirably be accurately and precisely controlled depending on a degree of insufflation of the target site (e.g., depending on an amount of insufflation gas such as carbon dioxide introduced via an endoscope or otherwise to or proximate to the target site). Insufflation such as with carbon dioxide gas can cause the radial expansion of tissue at a target site, thereby improving visibility of target tissue at the target site. This radial expansion of the tissue can impart a radial expansive force at least on the connecting structure 116 (e.g., a plurality of filaments formed of shape memory or elastomeric material). While the connecting structure may expand to a degree radially, at least a portion of the radial expansive force can also cause increased lift of the target tissue thereby permitting the selective lifting of the target tissue based on a degree of insufflation of the target site. Moreover, use of the connecting structure 116 that is formed of a shape memory material or elastomeric material can promote other aspects herein such as permitting the tissue traction devices herein to be readily coupled to opposing tissue walls of the anatomical wall having different diameters (e.g., due to the use of the flexible shape memory or elastomeric material).

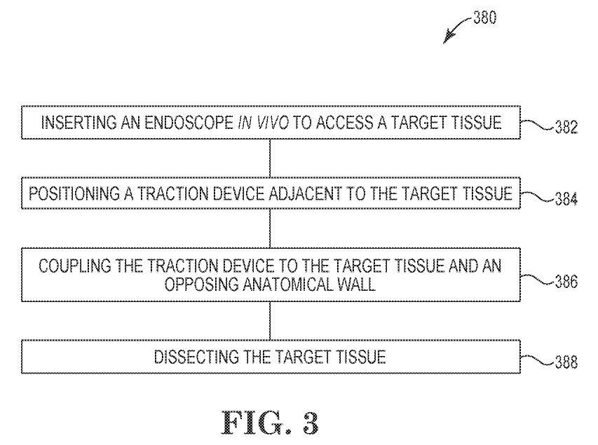

FIG. 3 provides an example of a method 380 of endoscopic submucosal dissection according to an embodiment of the disclosure. The method 380 can be employed with the endoscopes and tissue traction devices, described herein. At 382, the method 380 can include inserting an endoscope in vivo to access a target tissue (e.g., a lesion) in a patient. For instance, the target lesion or target tissue can be located in an esophagus of a patient, as described herein.

At 384, the method 380 can position the tissue traction device adjacent to the target tissue. For instance, the tissue traction device can be distally advanced through a working channel of the endoscope until the tissue traction device is adjacent to the target tissue. The tissue traction device can be distally advanced while in a constrained or delivery configuration (e.g., have a relatively small radial profile). The tissue traction device can be coupled to a distal end or other portion of an elongate wire, tube or projection that is advanced distally through the working channel of an endoscope until the tissue traction device is positioned at or adjacent to the target tissue. In some embodiments, the tissue traction device can be contained within a lumen of the endoscope or a lumen of a tube or catheter disposed in a lumen of the endoscope.

At 386, the method 380 can include coupling the tissue traction device to tissue at the target site. For instance, a tissue engagement portion of the tissue traction device can be coupled to tissue at a target site. For instance, both a first tissue engagement portion and second tissue engagement portion can be coupled to tissue at the target site (e.g., target tissue and tissue on a surface opposing the target tissue, respectively).

The tissue engagement portions can be coupled to the tissue at the target site via one or more coupling mechanisms. The coupling mechanisms can include mechanical anchors and/or an adhesive, as described herein. In some embodiments, the coupling mechanism can be positioned on the outer surfaces (e.g., the radial-most surfaces) of each of the tissue engagement portions to facilitate coupling of the tissue engagement portions to the tissue at the target site. For example, the coupling mechanisms can be integral with the tissue engagement portions or can be applied to the tissue engagement portions (e.g., ex vivo before the tissue traction device is delivered in vivo to the target site). In some cases, the coupling mechanisms such as mechanical anchors may be formed from the same material as the tissue engagement portions. Having the mechanical anchors be formed of the same material as the tissue engagement portions can promoting reliable and effective coupling of the mechanical anchors to the tissue at the target site. Alternatively, the coupling mechanisms can be formed of a different type of material than the tissue engagement portions. For instance, the coupling mechanisms can be a mechanical anchor or adhesive that the that is applied to the outer surfaces of the tissue engagement portions before or after the tissue traction device is delivered to the target site.

In some embodiments, force (e.g., radial force) may be applied to one or more of the tissue engagement portions (e.g., by applying force to an inner surface of the tissue engagement portions) to promote coupling of the coupling mechanisms to the tissue and/or to promote imparting a lifting force on the target tissue. The force can be applied with the endoscope, a tool (e.g., balloon catheter, etc.) extending through the endoscope, and/or an insufflation gas (e.g., carbon dioxide). These are merely examples and other mechanisms of coupling or promoting imparting lifting force on the target tissue are possible. Alternatively or additionally, the tissue traction device can be formed of a shape memory material that is configured to expand radially to a predetermined shape (e.g., a V-shape) once delivered to the target site in vivo.

In any case, a first tissue engagement portion of the tissue traction devices herein can be coupled to the target lesion at the target site and the second tissue engagement portion can be coupled to an opposing anatomical wall (a radially opposed wall relative to the target lesion) at the target site. It should be appreciated that the “force” stored within or imparted by the materials (e.g., the connecting structure 116) of the tissue traction devices herein allow the tissue traction devices herein to apply and maintain constant upward (e.g., radial) lifting/retraction pressure against the target tissue. That is, the natural tendency of the tissue traction device to move (i.e., have the connecting structure configured to contract the first tissue engagement portion radially toward the second tissue engagement portion) allows the target tissue to be lifted or elevated above the cutting plane such that the non-dissected target tissue portion may be more easily visualized and more efficiently excised.

After coupling the tissue engagement portions to the tissue at the target site, the method 380 can include dissecting or removing the target tissue (e.g., a target lesion), as indicated at 388. For example, a cutting element (e.g., cutting element 140 as illustrated in FIG. 1B) may be advanced through a working channel of an endoscope to dissect the target tissue (e.g., while lifted or under radial tension imparted by the tissue traction device) from the surrounding healthy tissue. Due to the configuration of the tissue traction devices herein, the target tissue can remain under tension or lifted until most or all of it is dissected. Hence, the tissue traction device described herein, and the methods of using endoscopes in conjunction with these tissue traction devices, can provide improved control over the degree of tension imparted on a target lesion. This can include predetermined tensioning based on the tendency of the shape memory or elastomeric material of the connecting structure to contract radially once the device is coupled to tissue at a target site, or variable tensioning of the target lesion depending on the amount of radial contraction of the tissue traction device relative to the degree of insufflation at the target site. The tissue tension devices and methods of use thereof ensures that the target lesion remains under tension before and during dissection by the endoscope's cutting member, unlike previous methods that relied on clips. Moreover, and unlike previous approaches such as those that employ clips, the method 380 can include retracting the entire endoscope and all components associated with the method 380 subsequent to removal of the target lesion. For instance, the method 380 can include removal of all elements of the tissue traction devices herein after completing dissection of the target lesion. Thus, the tissue traction devices and methods of use thereof (e.g., method 380) can result in the effective removal (dissection) of the target lesion, and yet leaves no foreign materials (e.g., clips) left in the patient after retracting the endoscope from the patient.

Once the target tissue is fully dissected, it may be removed for biopsy through a working channel of the endoscope using a suitable grasping element (not depicted) and/or by way of retrieval of the tissue traction device (e.g., to which some or all of the tissue of the target lesion remains coupled after dissection). Alternatively, some or all of the dissected target tissue may be left within the body passage for natural removal.

In various embodiments, the endoscopic instrument of the present disclosure is not limited to a tissue cutting element (e.g., electrocautery knife, etc.), but may include a variety of medical instruments configured to manipulate a target tissue (e.g., ablative elements, needles or syringes configured to inject agents into the target tissue, etc.). In addition, it should be appreciated that various elements herein such as the tissue traction devices and the endoscopic instrument of the present disclosure are not necessarily drawn to scale, but may be represented in a somewhat enlarged configuration to provide the requisite level of detail to understand and practice the embodiments of the present disclosure.

All of the devices and/or methods disclosed and claimed herein can be made and executed without undue experimentation in light of the present disclosure. While the devices and methods of this disclosure have been described in terms of preferred embodiments, it will be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A tissue traction device comprising:
a first tissue engagement portion configured to be coupled to a target tissue at an in-vivo target site;
a second tissue engagement portion configured to be coupled to an anatomical wall opposing the target tissue; and
a connecting structure located between and coupled to or integral with the first tissue engagement portion and the second tissue engagement portion.

2. The tissue traction device of claim 1, further comprising:
a first coupling mechanism on the first tissue engagement portion, wherein the first coupling mechanism is configured to couple a surface of the first tissue engagement portion to the target tissue; and
a second coupling mechanism on the second tissue engagement portion, wherein the second coupling mechanism is configured to couple a surface of the second tissue engagement portion to the anatomical wall opposing the target tissue.

3. The tissue traction device of claim 2, wherein the first coupling mechanism, the second coupling mechanism, or both the first coupling mechanism and the second coupling mechanism are an adhesive.

4. The tissue traction device of claim 2, wherein the first coupling mechanism, the second coupling mechanism, or both the first coupling mechanism and the second coupling mechanism are a mechanical anchor.

5. The tissue traction device of claim 4, wherein the mechanical anchor is a tine, fork, hook, finger, barb, or any combination thereof.

6. The tissue traction device of claim 1, wherein the tissue traction device is configured to create tissue traction between the target tissue and the anatomical wall opposing the target tissue when the tissue traction device is coupled to the target tissue and the anatomical wall opposing the target tissue.

7. The tissue traction device of claim 1, wherein:
the first tissue engagement portion is a first planar patch; and
the second tissue engagement portion is a second planar patch.

8. The tissue traction device of claim 7, wherein the connecting structure is a substantially radially extending member that is coupled to opposing surfaces of the first planar patch and the second planar patch.

9. The tissue traction device of claim 8, wherein the substantially radially extending member is formed of a shape memory material or an elastomeric material.

10. The tissue traction device of claim 9, wherein the substantially radially extending member is included in a plurality of substantially radially extending members that are disposed at respective positions between the opposing surfaces of the first planar patch and the second planar patch.

11. The tissue traction device of claim 9, wherein the substantially radially extending member comprises one or more substantially radially extending filaments or strips of material.

12. The tissue traction device of claim 1, wherein tissue traction device is a monolithic structure including the first tissue engagement portion, the second tissue engagement portion, and the connecting structure.

13. The tissue traction device of claim 12, wherein the tissue traction device is formed of a shape memory material.

14. The tissue traction device of claim 12, wherein:
the first tissue engagement portion is located at a first end region of the monolithic structure; and
the second tissue engagement portion is located at a second end region of the monolithic structure that opposes the first end region.

15. The tissue traction device of claim 12, wherein the tissue traction device is a V-shaped, C-shaped, U-shaped, or Y-shaped monolithic structure.

16. A tissue traction device comprising:
a first tissue engagement portion configured to be coupled to a target tissue at an in-vivo target site;
a first coupling mechanism coupled to or integral with the first tissue engagement portion;
a second tissue engagement portion configured to be coupled to an anatomical wall opposing the target tissue;
a second coupling mechanism coupled to or integral with the second tissue engagement portion; and
an individual connecting structure that is integral with and located between the first and second tissue engagement portions.

17. The tissue traction device of claim 16, wherein the tissue traction device is a monolithic structure including the first tissue engagement portion, the second tissue engagement portion, and the individual connecting structure.

18. The tissue traction device of claim 17, wherein the monolithic structure is V-shaped.

19. A tissue traction device comprising:
a first tissue engagement portion configured to be coupled to a target tissue at an in-vivo target site;
a first coupling mechanism coupled to or integral with the first tissue engagement portion;
a second tissue engagement portion configured to be coupled to an anatomical wall opposing the target tissue;
a second coupling mechanism coupled to or integral with the second tissue engagement portion; and
a plurality of connecting structures coupled to and located between the first tissue engagement portion and the second tissue engagement portion, wherein each connecting structure of the plurality of connecting structures is a substantially radially extending filament or strip of material that is configured to impart a variable lifting force on the target tissue in response to insufflation of the target site.

20. The tissue traction device of claim 19, wherein the substantially radially extending connecting structures are spaced apart along a longitudinal axis of the tissue traction device.
